(19) United States
(12) Patent Application Publication
(10) Pub. No.: US 2021/0077643 A1
(43) Pub. Date: Mar. 18, 2021

(54) MODULAR ULTRAVIOLET (UV) STERILIZATION LIGHTING ASSEMBLIES

(71) Applicant: Harbor Innovations, LLC, Oyster Bay, NY (US)

(72) Inventors: Rachel Dombrowsky, Hewlett, NY (US); Joseph Toro, Hauppauge, NY (US)

(21) Appl. No.: 17/022,970

(22) Filed: Sep. 16, 2020

Related U.S. Application Data
(60) Provisional application No. 62/900,932, filed on Sep. 16, 2019.

(51) Int. Cl. A61L 2/10; A61L 2/26; A61L 9/20; B08B 7/00; A47L 9/28; A47L 9/30

(52) U.S. Cl. CPC A61L 2/10; A61L 2/26; A61L 9/20; B08B 7/0035; A47L 9/2852; A47L 2201/04; A47L 9/2826; A61L 2202/11; A61L 2202/17; A61L 2209/12; A61L 2209/14; A47L 9/30

(57) ABSTRACT

An ultraviolet (UV) sterilization lighting system includes a plurality of arrays of UV sterilization lighting and a power source. The arrays of UV sterilization lighting are configured in a modular arrangement in electrical communication with the power source and enable plug-in type completion of electrical communication within the arrays. The arrays are configured in a pattern to effect desired sterilization via the UV sterilization lighting. The array formations include straight and/or curved lines, circles, triangles, squares, rectangles, pentagons, hexagons and other similar polygonal or other geometrical arrangements, whether in an ordered or disordered pattern, that are deemed most suitable to affect the desired sterilization.

### CROSS-REFERENCE TO RELATED APPLICATION

This U.S. Non-Provisional Patent Application claims priority to U.S. Provisional Patent Application No. 62/900,932, filed on Sep. 16, 2019, the disclosure of which is incorporated by reference herein in its entirety.

### TECHNICAL FIELD

This disclosure relates to sanitizing equipment, and more particularly, to sanitizing equipment utilizing ultraviolet light to destroy pathogens.

### BACKGROUND

Many indoor and outdoor surfaces contain a variety of pathogens. As individuals and devices traverse these surfaces, they pick up and carry pathogens that can cause sickness, disease, and possibly death. For example, the recent appearance of the virus SARS-CoV-2 has resulted in a need for frequent sterilization of both indoor and outdoor surfaces in a plethora of environments, often at multiple times within a single day.

### SUMMARY

This disclosure is directed to modular sterilization assemblies and/or systems that use ultraviolet light to destroy or inhibit the growth of surface pathogens, such as, for example, virus, bacteria, mold, spore, and fungi, and/or to reduce chemical contaminants. In particular, the modular sterilization assemblies include an array of light or energy emitting devices (e.g., UV LED's) that emit short-wavelength ultraviolet (“UV-C”) light directed at building surfaces such as hospitals, nursing homes, assisted living or the like to destroy pathogens that may be associated therewith.

In embodiments, modular UV sterilization systems of this disclosure include a proximity sensor that controls when the UV LEDs are powered on and a warning light to warn persons of active UV cleaning components. The system may include a UV module, a proximity sensor, AC power, shrouds, mounting brackets tailored to applications, robotic mounts, and a computer application or “app” on a mobile phone, for example, to control and/or monitor the system, set cleaning parameters, such as time of day, areas to clean, etc.

In an aspect of the disclosure, an ultraviolet (UV) sterilization lighting system includes arrays of UV sterilization lighting and a power source. The arrays of UV sterilization lighting are in a modular arrangement and in electrical communication with the power source. The arrays of UV sterilization lighting are arranged to enable plug-in type completion of electrical communication within the arrays. The arrays are configured in a pattern to effect desired sterilization via the UV sterilization lighting.

In some aspects of the disclosure, the arrays of UV sterilization lighting may be configured in at least one formation selected from a straight line, a curved line, a circle, a triangle, a square, a rectangle, or a polygon. The at least one formation may be in at least one of an ordered pattern or a disordered pattern.

In some aspects of the disclosure, the modular arrangement of the arrays of UV sterilization lighting may be in electrical communication with at least one cleaning device. The cleaning device may include a computing device including a processor and a memory. The memory stores instructions which, when executed by the processor, cause the computing device to control the UV sterilization lighting system. The UV sterilization lighting system may be in photonic communication with objects being cleaned. The UV sterilization lighting system may be configured to signal a user of the system that the robotic cleaning device is in operation in proximity to the user.

In some aspects of the disclosure, the modular arrangement of the arrays of UV sterilization lighting may be disposed in a shroud that directs UV sterilization lighting emitted therefrom in a direction toward articles of clothing or footwear of a person.

In some aspects of the disclosure, the cleaning device may include a rotatable assembly that includes a first rotating member coupled to a second rotating member that enables operation and aiming of a UV sterilization lighting array disposed on the second rotating member. The UV sterilization lighting array may be disposed on the second rotating member included in the arrays of UV sterilization lighting configured in a modular arrangement.

In some aspects of the disclosure, the modular arrangement of the arrays of UV sterilization lighting may be disposed in a heating, ventilating and air conditioning (HVAC) filter cartridge configured with UV sterilization lighting arrays positioned within the cartridge to enable sterilization of air flow through the cartridge.

In another aspect of the disclosure, an UV light sterilization device includes a mobile cleaning device. At least one array of UV lights is disposed about the mobile cleaning device. UV lights of the at least one array of UV lights are configured to be selectively activated to emit UV light to degrade pathogens. A power source is disposed about the mobile cleaning device. The power source is in electrical communication with the at least one array of UV lights. Activation of the power source activates the at least one array of UV lights. A computing device is in communication with the mobile cleaning device. The computing device includes a processor and a memory. The computing device controls the power source to activate the at least one array of UV lights to sterilize a space in which the mobile cleaning device is located by degrading pathogens.

In some aspects of the disclosure, at least two arrays of UV lights may be arranged in a modular configuration about the mobile cleaning device. The at least two arrays of UV lights may be arranged to sterilize at least one of a floor, a wall, a ceiling, or an object positioned in the space in which the mobile cleaning device is located.

In some aspects of the disclosure, the mobile cleaning device may include a proximity sensor configured to detect movement of an object or person within a predetermined distance from the mobile cleaning device. The mobile cleaning device may include a warning light configured to be activated when the object or person is detected within the predetermined distance from the mobile cleaning device.

In some aspects of the disclosure, a motor may be configured to rotate the at least one array of UV lights.

In some aspects of the disclosure, the computing device may communicate with the mobile cleaning device via a wireless signal.

In some aspects of the disclosure, the UV lights of the at least one array of UV lights may be Light Emitting Diodes (LEDs).

In some aspects, the mobile cleaning device may be a self-mobile robotic vacuum cleaner.

In some aspects of the disclosure, the computing device may be a smartphone, tablet, laptop computer, or desktop computer.

In yet another aspect of the disclosure, a method of sterilizing a space using UV light includes providing a device including a mobile cleaning device having at least one array of UV lights configured to be selectively activated to emit UV light to degrade pathogens. The method includes scanning, by a scanning device of the mobile cleaning device, a space in which the mobile cleaning device is located. The method includes determining, by the computing device, a series of conceptual physical nodes within the space in which the mobile cleaning device is located. The method includes calculating, by the computing device, a shortest pathway from an origin node of the series of conceptual physical nodes, to each of the other conceptual physical nodes, and back to the origin node. The method includes determining, by the computing device, an activation schedule of the at least one array of UV lights for when the mobile cleaning device is located at each of the conceptual physical nodes. The method includes traversing the mobile cleaning device along the shortest pathway from the origin node, and activating the at least one array of UV lights at each node of the plurality of conceptual physical nodes according to the activation schedule to degrade pathogens in the space in which the mobile cleaning device is located.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1A illustrates various embodiments of modular UV sterilization lighting systems and assemblies in accordance with aspects of the disclosure;

FIG. 1B illustrates an exemplary array of UV lights in the system of FIG. 1A;

FIG. 1C illustrates an exemplary self-piloting vacuum device in the system of FIG. 1A;

FIG. 1D illustrates an exemplary HVAC filter cartridge in the system of FIG. 1A;

FIG. 2 illustrates an UV light sterilization device in accordance with aspects of the disclosure;

FIG. 3 illustrates an exemplary UV light sterilization pathway of a mobile cleaning device in accordance with aspects of the disclosure;

FIG. 4 is a flowchart of a method of sterilizing a space using UV light in accordance with aspects of the disclosure; and

FIG. 5 illustrates an exemplary computer system in accordance with aspects of the disclosure.

### DETAILED DESCRIPTION

Exemplary embodiments of the disclosed systems, devices, methods, and assemblies are described in detail with reference to the drawings, in which like reference numerals designate identical or corresponding elements in each of the several views. As used herein, the terms parallel and perpendicular are understood to include relative configurations that are substantially parallel and substantially perpendicular up to about +or −10 degrees from true parallel and true perpendicular.

In the following description, well-known functions or constructions are not described in detail to avoid obscuring the disclosure in unnecessary detail.

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

[symbol]

This disclosure relates to a suite of products centered on a modular UV LED panel that allow sterilization to be performed in various methods and for different field situations and conditions.

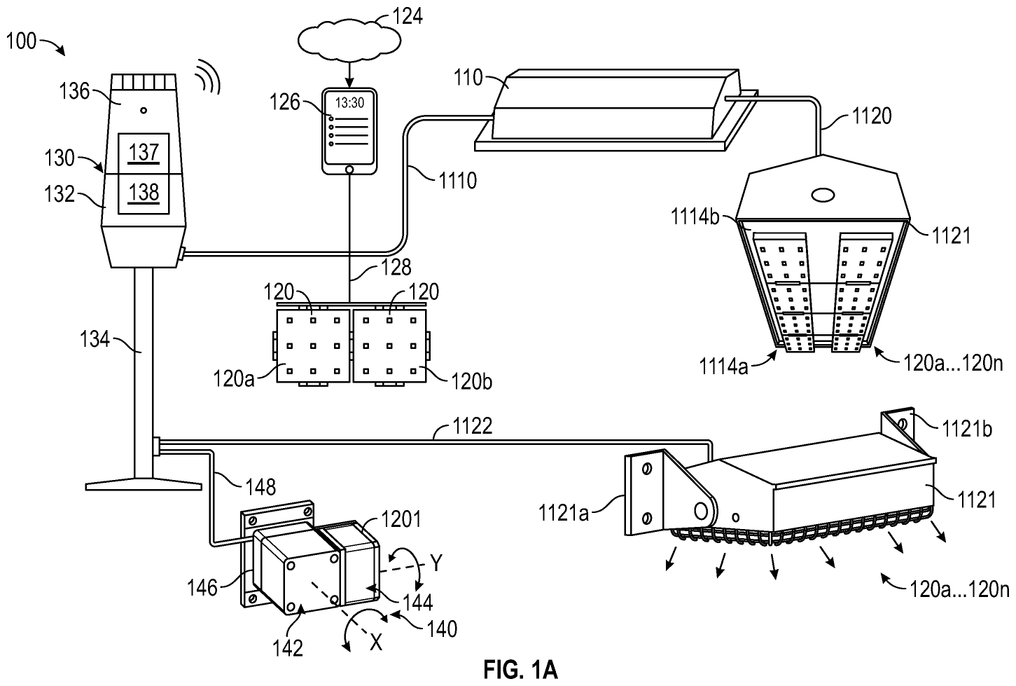

FIG. 1A illustrates an ultraviolet lighting sterilization system 100 that includes modular ultraviolet (UV) sterilization lighting assemblies 120. The lighting assemblies 120 include light-emitting diodes, that are configured for photonic and electrical communication with equipment such as equipment utilized for medical cleaning, filtration, illumination, and other applications according to this disclosure. The modular ultraviolet (UV) sterilization lighting assemblies 120 are configured to emit ultraviolet light, such as short-wavelength ultraviolet (“UV-C”) light, including in the form of light-emitting diodes (LEDs) selectively and permanently attached to such equipment for use in a medical facility such as, for example, a hospital, nursing home, assisted living or the like.

The systems 100 and assemblies 120 according to this disclosure are configured to enable plug-in type completion of electrical communication, whether by wire or wirelessly, in various array formations of the UV lighting LEDs, including straight and/or curved lines, circles, triangles, squares, rectangles, pentagons, hexagons and other similar polygonal or other geometrical arrangements, whether in an ordered or disordered pattern, that are deemed most suitable to effect the desired sterilization.

The phrases “computing device” and “electronic controller” and “controller” may be used interchangeably herein.

More particularly, system 100 includes an AC/DC power supply 110 that provides AC power through a cord 1110 and/or DC power through a cord 1120. In an aspect of the disclosure, AC power cord 1110 is electrically coupled to a cleaning device 130 such as a vacuum cleaning device or a floor polisher or the like. In embodiments, the cleaning device 130 may be battery-powered. Such cleaning devices 130 include a motor and suction canister 132 pneumatically coupled to a cleaning conduit 134. The motor and suction canister 132 can include a processor 136 having memory. The processor/memory 136 may be in electromagnetic communication via signals 136′ with a cloud or local server 124 that stores instructions in memory for an electronic controller or computing device 126. The processor/memory 136 may be fixed on the cleaning device 130 or on the systems and assemblies 120. The electronic controller 126 can be manually operated by a user (not shown). The cleaning conduit 134 can include a proximity sensor 137 and warning light 138. An example of a vacuum cleaning device that may be utilized is a Dyson V7 Motorhead Cord-free Stick Vacuum by Dyson Mfg. Chicago, Ill., USA. Alternatively, the cleaning device 130 may be a self-mobile robotic vacuum cleaner.

In an aspect of the disclosure, the electronic controller 126 is operably coupled to a modular UV sterilization lighting assembly 120 that includes at least two arrays of UV sterilization lighting 120a and 120b that are supplied power by wire connection 128 or by a battery (not shown).

In an aspect of the disclosure, the modular UV sterilization assembly 120 may include additional arrays of UV sterilization lighting 120a . . . 120n (see, e.g., FIG. 1B). The arrays of UV sterilization lighting 120a . . . 120n may be mounted in a shroud 1121 that is in electrical communication with the ac/dc power supply 110 via the DC power cord 1120. Operation of the arrays of UV sterilization lighting 120a . . . 120n is controlled by the electronic controller 126 receiving a signal from the proximity sensor 137 via the processor 136.

The AC/DC power supply 110 i) may be supplied with power externally from electrical distribution wiring within the medical facility or ii) may be powered internally via a selectively removable power supply such as a battery or capacitor or the like or iii) may receive wireless power.

The shroud 1121 may include mounting brackets 1121a and 1121b for positioning of the shroud 1121 and the arrays of UV sterilization lighting 120a . . . 120n.

The systems and assemblies 120 may be configured to enable electrical communication between the arrays 120a . . . 120n by insertion in a plug-in type manner on a front portion 1114a of the shroud 1121 or on a rear portion 1114b of the shroud 1121.

As the cleaning device 130 moves within its operating environs such as a medical facility workspace or patient confinement area, or other area of operation, the arrays of sterilization lighting 120a . . . 120n are actuated by the processor 136 of the cleaning device 130 autonomously such as via sensors, by a trigger actuator, or by a switch.

The proximity sensor 137 and warning light 138 control when the UV LEDs are powered on and warn persons of active UV cleaning components.

Robotic operating features may include rotatable assembly 140 that includes a first rotating member 142 that is rotatable around axis x-x via a motor 146 in electrical communication with the vacuum 130 via power cord 148. A second rotating member 144 is coupled to the first rotating member 142 and rotates with the first rotating member 142 around axis x-x and is rotatable around an axis y-y that is perpendicular to axis x-x to enable operation and aiming of a UV sterilization lighting array 1201 disposed on the second rotating member 144 to clean walls or floors.

The vacuum cleaning device 130 may also provide electrical power directly to the shroud 1121 via power cord 1122 that is in electrical communication with the device 130 through the cleaning conduit 134.

In addition to, or in lieu of, the canister type cleaning device 130, the UV lighting sterilization system 100 may include a self-piloting vacuum device 150 (see, e.g., FIG. 1C) that is configured to enable mounting of UV sterilization lighting array 1201 for operating and aiming of the array 1201.

In an aspect of the disclosure, a heating, ventilating, and air conditioning (HVAC) filter cartridge 160 may be configured with UV sterilization lighting arrays 162 positioned within the cartridge 160 to enable sterilization of air flow through the cartridge 160 (see, e.g., FIG. 1D). As understood by those skilled in the art, power to the UV sterilization lighting arrays 162 may be provided by routing of wiring from AC/DC power supply 110 that provides AC power through cord 1110 and/or DC power through cord 1120. Such UV sterilization lighting arrays 162 may facilitate preventing the outbreak of airborne diseases such as Legionnaire's disease or the like.

Securement of any of the components of the disclosed devices of the UV sterilization lighting assembly system 100 may be effectuated using known securement techniques such welding, crimping, gluing, heat-shrinking, fastening, etc.

Wherever technically feasible, UV light sterilization system 200 may include substantially the same features as the UV light sterilization system 100 described herein, unless otherwise indicated. For example, the mobile cleaning device 230 described herein may include substantially the same features as the cleaning device 130 described herein, unless otherwise indicated.

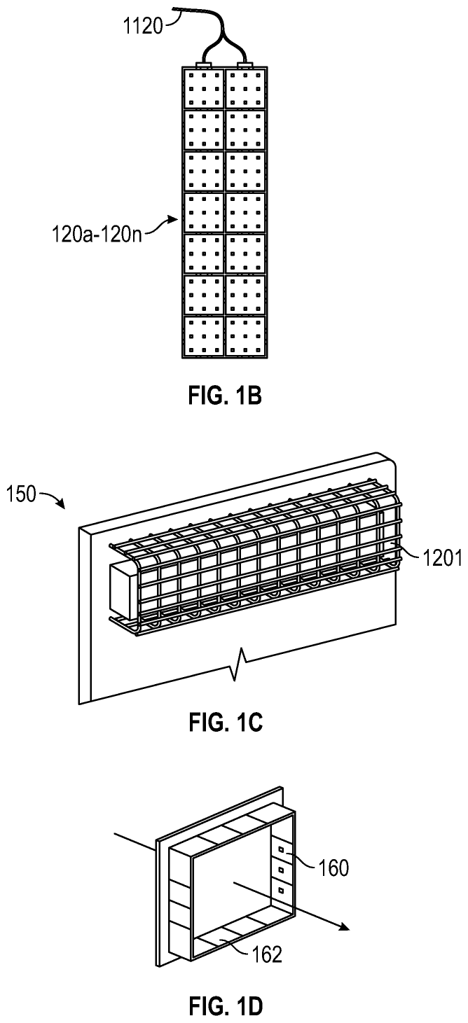

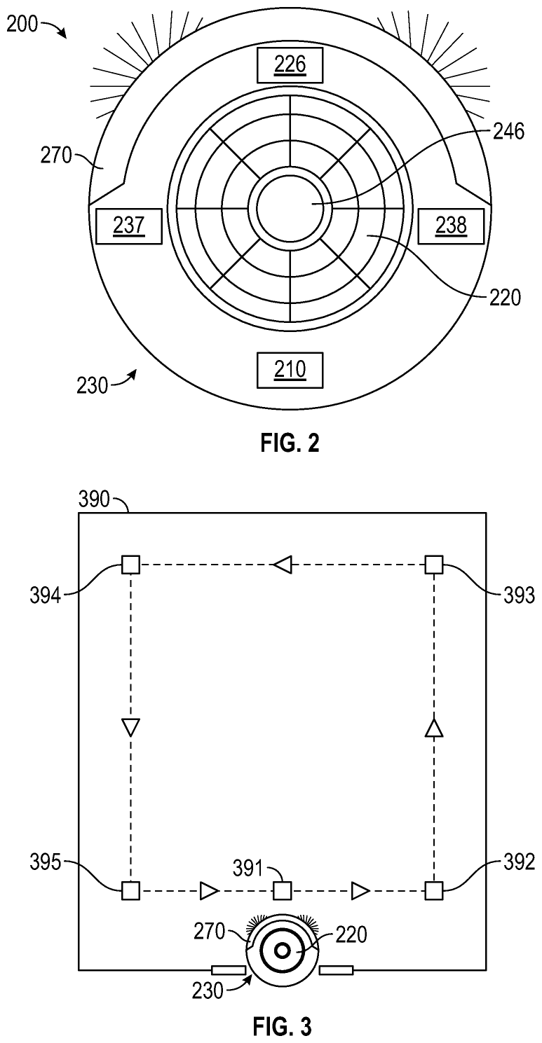

Referring to FIGS. 2 to 3, a UV light sterilization system 200 includes a mobile cleaning device 230. The UV light sterilization system 200 may be embodied in the mobile cleaning device 230. At least one array of UV lights 220 is disposed about the mobile cleaning device 230. Any of the arrays of UV lights described with reference to FIGS. 1A-1D may be employed by the mobile cleaning device 230. UV lights of the at least one array of UV lights 220 are configured to be selectively activated to emit UV light to degrade pathogens. For example, the UV lights can be controlled such that each individual light in an array can be selectively activated for an individual period of time and at individually controlled levels of intensity. Thus, less than all of the UV lights in an array can be activated, and any number of lights in an array can be selectively and dynamically activated at individual levels of intensity (e.g., by receiving individualized levels of electrical power from power source 210). The power source 210 is disposed about the mobile cleaning device 230. The power source 210 is in electrical communication with the at least one array of UV lights 220. Activation of the power source 210 activates the at least one array of UV lights 220. A computing device 226 is in communication with the mobile cleaning device 230 (e.g., may be disposed about the mobile cleaning device 230 or may be remotely positioned with respect to the mobile cleaning device 230). The computing device 226 includes a processor and a memory, as described herein. The computing device 226 controls the power source 210 to activate the at least one array of UV lights 220 to degrade pathogens and sterilize a space 390 in which the mobile cleaning device 230 is located.

In some aspects of the disclosure, at least two arrays of UV lights (see, e.g., 120a and 120b in FIG. 1A) are arranged in a modular configuration about the mobile cleaning device 230. The at least two arrays of UV lights are arranged to sterilize at least one of a floor, a wall, a ceiling, or an object positioned in the space 390 in which the mobile cleaning device 230 is located.

In some aspects of the disclosure, the mobile cleaning device 230 includes a proximity sensor 238 configured to detect movement of an object or person within a predetermined distance from the mobile cleaning device 230. The mobile cleaning device 230 includes a warning light 237 configured to be activated when the object or person is detected within the predetermined distance from the mobile cleaning device 230 by the proximity sensor 238.

In some aspects of the disclosure, a motor 246 is configured to rotate the at least one array of UV lights 220, e.g., UV LEDs. For example, the motor 246 may elevate, rotate, invert, tilt, and otherwise manipulate the at least one array of UV lights 220 to face in any direction, thus enabling the mobile cleaning device 230 to clean objects or surfaces in any special relationship with the mobile cleaning device. As an example, UV light may be emitted in all directions in which an outer surface of a sphere would face.

In some aspects of the disclosure, the computing device 226 communicates with the mobile cleaning device 230 via a wireless signal (e.g., when the computing device 226 is remotely positioned with respect to the mobile cleaning device 230).

In some aspects of the disclosure, the UV lights of the at least one array of UV lights 220 are Light Emitting Diodes (LEDs).

In some aspects of the disclosure, the mobile cleaning device 230 is a self-mobile robotic vacuum cleaner.

In some aspects of the disclosure, the computing device 226 is a smartphone, tablet, laptop computer, or desktop computer. The computing device 226 may be positioned about the mobile cleaning device 230, or may be in a remote location and communicate wirelessly with the mobile cleaning device (e.g., via WiFi, Bluetooth, cell network, or cloud network based communication).

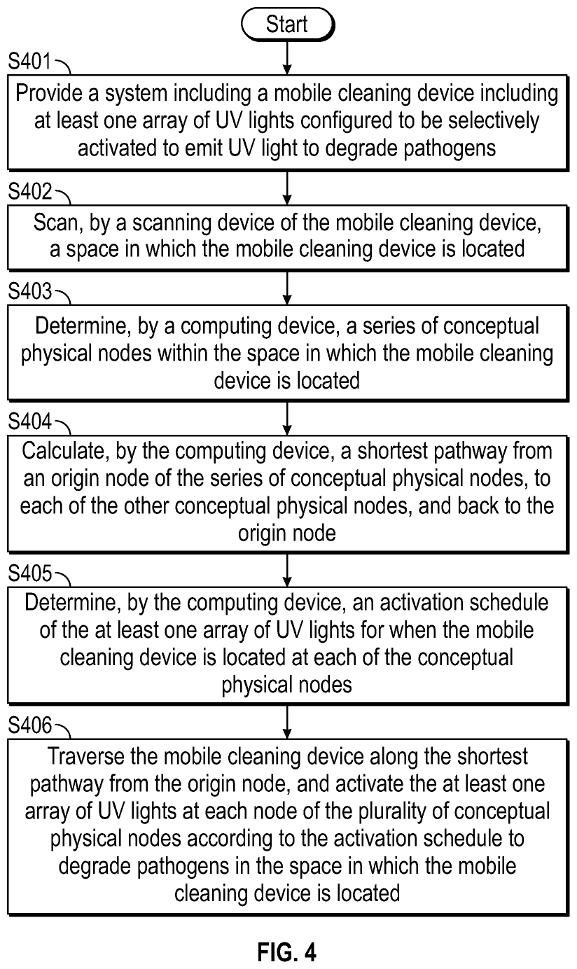

Referring to FIGS. 3 and 4, a method of sterilizing a space using UV light includes (S401) providing a system including a mobile cleaning device 230 including at least one array of UV lights 220 configured to be selectively activated to emit UV light to degrade pathogens. The method includes (S402) scanning, by a scanning device 270 of the mobile cleaning device 230, a space 390 in which the mobile cleaning device 230 is located. The method includes (S403) determining, by the computing device 226, a series of conceptual physical nodes (e.g., 391, 392, 393, 394, and 395) within the space 390 in which the mobile cleaning device 230 is located.

The method includes (S404) calculating, by the computing device 226, a shortest pathway from an origin node 391 of the series of conceptual physical nodes, to each of the other conceptual physical nodes (e.g., 392, 393, 3954, and 395), and back to the origin node 391. The method includes (S405) determining, by the computing device 226, an activation schedule of the at least one array of UV lights 220 for when the mobile cleaning device 230 is located at each of the conceptual physical nodes, and/or when the mobile cleaning device 230 is moving between the conceptual physical nodes. The method includes (S406) traversing the mobile cleaning device 230 along the shortest pathway from the origin node 391 to allow UV light sterilization of the space 390, and activating the at least one array of UV lights 220 at each node of the plurality of conceptual physical nodes (e.g., 392, 393, 394, and 395) according to the activation schedule to degrade pathogens in the space 390 in which the mobile cleaning device 230 is located.

The activation schedule of the at least one array of UV lights 220 may include aiming the at least one array of UV lights, or multiple arrays of UV lights in different directions to direct UV light to a surface, object, or area to be sterilized. The activation schedule includes activating the UV lights for a predetermined period of time at each conceptual physical node. The UV lights may also be illuminated while the mobile cleaning device 230 is in-transit from one physical node to another.

Each conceptual physical node corresponds with a physical location in the space 390 in which the mobile cleaning device is located. For example, a pathway may be traversed around a perimeter of the space 390 to allow sterilization of substantially the entire space 390, including all objects positioned therein. As an example, the activation schedule may include a dynamic pathway in which particular areas of interest are cleaned more frequently or for a longer period of time than other areas. For example, particularly high traffic areas may be sterilized more often and/or more intensely than low traffic areas. Additionally, the system described herein may schedule sterilization procedures to be carried out at times when certain areas are not in use, such as overnight or when an area is not scheduled to be occupied or used. A plurality of sensors positioned throughout a space or building can be used to record foot traffic and times in which a particular area is occupied, and machine learning algorithms described herein may be employed to generate an activation schedule that maximizes convenience to users of a building or space.

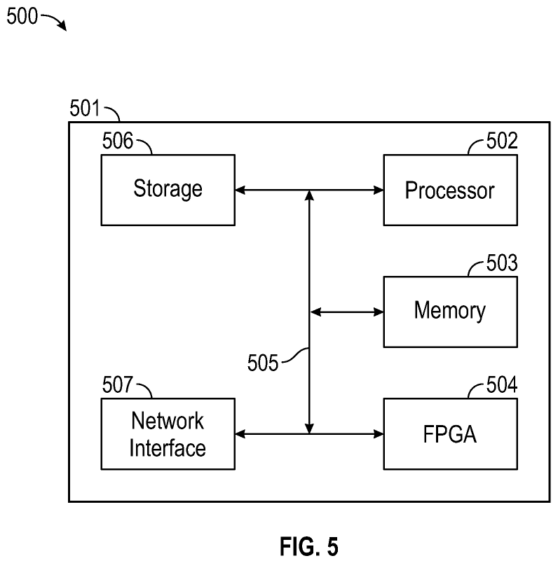

Referring to FIG. 5, a computer 500 employed by the UV light sterilization system (e.g., 100 or 200), such as a computer employed by the computing device 126, or a computer employed by the cleaning device 130 is generally described. As an example, the computing device 126 may be a smartphone, tablet computer, or a laptop computer. The software or algorithms described herein may be included in or employed by a smartphone or tablet application, for example.

The computer 500 may include a control unit 501. The control unit 501 may include a processor 502 connected to a computer-readable storage medium or a memory 503 which may be a volatile type memory, e.g., RAM, or a non-volatile type memory, e.g., flash media, disk media, etc. The processor 502 may be another type of processor such as, without limitation, a digital signal processor, a microprocessor, an ASIC, a graphics processing unit (GPU), field-programmable gate array (FPGA), or a central processing unit (CPU).

The memory 503 may store instructions, to be executed by the processor 502, to instruct the control unit 501 to receive (e.g., to continuously receive) data regarding the physical layout of a room or space (e.g., space 390) to be sterilized. The control unit 501 determines an ideal series of conceptual physical nodes (e.g., 391-395) and pathway therethrough within the space in which the mobile cleaning device 230 is located, and an ideal activation schedule of the array(s) of UV lights for sterilization the space 390.

According to an exemplary embodiment of the disclosure, the storage device 506 of the control unit 501 stores one or more machine learning algorithms and/or models, configured to determine an ideal series of conceptual physical nodes (e.g., 391-395) and pathway therethrough within the space 390 in which the mobile cleaning device 230 is located, and an ideal activation schedule of the array(s) of UV lights. The machine learning algorithm may apply mathematical models to determine an ideal series of conceptual physical nodes and pathway therethrough within the space in which the mobile cleaning device 230 is located, and an ideal activation schedule of the array(s) of UV lights. The machine learning algorithm(s) may be trained on and learn from experimental data and/or data from previous spaces in which the mobile cleaning device 230 had sterilized to determine a sterilization procedure.

As an example, a “traveling salesman” algorithm based on the “traveling salesman problem” may be employed for plotting a course around a room or space to sterilize the room or space by activating a UV array described herein. The traveling salesman problem (TSP) asks the question: “Given a list of nodes in a geographic space and the distances between each pair of nodes, what is the shortest possible route that visits each node exactly once and returns to the origin node?” The solution to the “traveling salesmen problem” is the shortest combined route between each node to visit each node and arrive back at the origin node. For example, referring particularly to FIG. 3, in a square or rectangular room with 5 nodes, a square or rectangular pathway about the nodes in a clockwise or counterclockwise direction may be the solution to the TSP.

In various embodiments, the memory 503 can be random access memory, read-only memory, magnetic disk memory, solid state memory, optical disc memory, and/or another type of memory. The memory 503 can communicate with the processor 502 through communication buses 505 of a circuit board and/or through communication cables such as serial ATA cables or other types of cables. The memory 503 includes computer-readable instructions that are executable by the processor 502 to operate the control unit 501. The control unit 501 may include a network interface 507 to communicate with other computers or a server. A storage device 506 may be used for storing data. The control unit 501 may include one or more FPGAs 504. The FPGA 504 may be used for executing various machine learning algorithms such as those described herein.

Machine learning algorithms are advantageous for use in determining and applying an ideal series of conceptual physical nodes within the space in which the mobile cleaning device 230 is located, and an ideal activation schedule of the array(s) of UV lights, at least in that complex sensor components and pre-defined categorization rules and/or algorithms are not required. Rather, machine learning algorithms utilize the initially input data, e.g., the data of the geographic arrangement of the scanned space in which the mobile cleaning device 230 is located and/or correlations by analyzing data therefrom. Thus, with the one or more machine learning algorithms having been trained as detailed above, such can be used to determine an ideal series of conceptual physical nodes and pathway therethrough within the space in which the mobile cleaning device 230 is located, and an ideal activation schedule of the array(s) of UV lights.

As can be appreciated, the disclosed modular UV sterilization lighting assemblies and/or systems 100 can include any suitable electrical components to operate and/or communicate with the modular UV sterilization lighting assemblies and/or systems disclosed herein. Such electrical components can include, for example, one or more controllers and/or circuitry. As used herein, the term “controller” and like terms are used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device. The term “controller” also includes “processor,” “digital processing device” and like terms, and are used to indicate a microprocessor or central processing unit (CPU). The CPU is the electronic circuitry within a computer that carries out the instructions of a computer program by performing the basic arithmetic, logical, control and input/output (I/O operations specified by the instructions, and by way of non-limiting examples, include server computers. In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some embodiments, the operating system is provided by cloud computing.

In some embodiments, the controller includes a storage and/or memory device. The storage and/or memory device is one or more physical apparatus used to store data or programs on a temporary or permanent basis. In some embodiments, the controller includes volatile memory and requires power to maintain stored information. In some embodiments, the controller includes non-volatile memory and retains stored information when it is not powered. In some embodiments, the non-volatile memory includes flash memory. In some embodiments, the non-volatile memory includes dynamic random-access memory (DRAM). In some embodiments, the non-volatile memory includes ferroelectric random access memory (FRAM). In some embodiments, the non-volatile memory includes phase-change random access memory (PRAM). In some embodiments, the controller is a storage device including, by way of non-limiting examples, CD-ROMs, DVDs, flash memory devices, magnetic disk drives, magnetic tapes drives, optical disk drives, and cloud computing based storage. In some embodiments, the storage 506 and/or memory device 503 is a combination of devices such as those disclosed herein.

In some embodiments, the controller includes a display to send visual information to a user. In some embodiments, the display is a cathode ray tube (CRT). In some embodiments, the display is a liquid crystal display (LCD). In some embodiments, the display is a thin film transistor liquid crystal display (TFT-LCD). In some embodiments, the display is an organic light emitting diode (OLED) display. In various some embodiments, on OLED display is a passive-matrix OLED (PMOLED) or active-matrix OLED (AMOLED) display. In some embodiments, the display is a plasma display. In some embodiments, the display is a video projector. In some embodiments, the display is interactive (e.g., having a touch screen or a sensor such as a camera, a 3D sensor, a LiDAR, a radar, etc.) that can detect user interactions/gestures/responses and the like. In still some embodiments, the display is a combination of devices such as those disclosed herein.

As can be appreciated, the controller may include or be coupled to a server and/or a network. As used herein, the term “server” includes “computer server,” “central server,” “main server,” and like terms to indicate a computer or device on a network that manages the vehicle, vehicle components, and/or vehicle resources. As used herein, the term “network” can include any network technology including, for instance, a cellular data network, a wired network, a fiber optic network, a satellite network, and/or an IEEE 802.11a/b/g/n/ac wireless network, among others.

In some embodiments, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In embodiments, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the assemblies and/or systems.

The systems described herein may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that this disclosure is not limited to the precise embodiments described, and that various other changes and modifications may be affected by one skilled in the art without departing from the scope or spirit of this disclosure. Additionally, the elements and features shown or described in connection with certain embodiments may be combined with the elements and features of certain other embodiments without departing from the scope of this disclosure, and that such modifications and variations are also included within the scope of this disclosure. Accordingly, the subject matter of this disclosure is not limited by what has been particularly shown and described.

What is claimed is:

1. An ultraviolet (UV) sterilization lighting system, comprising:
a power source; and
a plurality of arrays of UV sterilization lighting configured in a modular arrangement in electrical communication with the power source,
wherein the modular arrangement of the plurality of arrays of UV sterilization lighting enable plug-in type completion of electrical communication between individual arrays of the plurality of arrays, the plurality of arrays configured in a pattern to effect sterilization via the UV sterilization lighting.

2. The system according to claim 1, wherein each array of the plurality of arrays is configured in at least one formation selected from the group consisting of (a) a straight line; (b) a curved line; (c) a circle; (d) a triangle; (e) a square; (f) a rectangle; and (g) a polygon.

3. The system according to claim 2, wherein the at least one formation is in at least one of an ordered pattern or a disordered pattern.

4. The system according to claim 1, wherein the modular arrangement of the plurality of arrays of UV sterilization lighting is in electrical communication with at least one cleaning device,
the at least one cleaning device including a computing device including a processor and a memory storing instructions which, when executed by the processor, cause the computing device to control the UV sterilization lighting system to be in photonic communication with objects being cleaned.

5. The system according to claim 1, wherein the modular arrangement of the plurality of arrays of UV sterilization lighting is disposed in a shroud.

6. The system according to claim 4, wherein the at least one cleaning device includes a rotatable assembly that includes a first rotating member coupled to a second rotating member that enables operation and aiming of at least one array of the plurality of arrays of UV sterilization lighting disposed on the second rotating member.

7. The system according to claim 1, wherein the modular arrangement of the plurality of arrays of UV sterilization lighting is disposed in a heating, ventilating or air conditioning (HVAC) filter cartridge to enable sterilization of air flow through the cartridge.

8. An ultraviolet (UV) light sterilization device, comprising:
a mobile cleaning device;
at least one array of UV lights disposed on the mobile cleaning device, the at least one array of UV lights configured to be selectively activated to emit UV light to degrade pathogens;
a power source disposed on the mobile cleaning device, the power source in electrical communication with the at least one array of UV lights, wherein activation of the power source activates the at least one array of UV lights; and
a computing device in communication with the mobile cleaning device, the computing device configured to control the power source to activate the at least one array of UV lights to sterilize a space in which the mobile cleaning device is located by degrading pathogens.

9. The device of claim 8, wherein at least two arrays of UV lights are arranged in a modular configuration about the mobile cleaning device, and wherein the at least two arrays of UV lights are arranged to sterilize at least one of a floor, a wall, a ceiling, or an object positioned in the space in which the mobile cleaning device is located.

10. The device of claim 8, wherein the mobile cleaning device includes a proximity sensor configured to detect movement of an object or person within a predetermined distance from the mobile cleaning device, and wherein the mobile cleaning device includes a warning light configured to be activated when the object or person is detected within the predetermined distance from the mobile cleaning device.

11. The device of claim 8, further including a motor configured to rotate the at least one array of UV lights.

12. The device of claim 8, wherein the computing device communicates with the mobile cleaning device via a wireless signal.

13. The device of claim 8, wherein the UV lights of the at least one array of UV lights are Light Emitting Diodes (LEDs).

14. The device of claim 8, wherein the mobile cleaning device is a self-mobile robotic vacuum cleaner.

15. The device of claim 8, wherein the computing device is a smartphone, tablet, laptop computer, or desktop computer.

16. A method of sterilizing a space using ultraviolet (UV) light, comprising:
locating a mobile cleaning device in a space to be cleaned;
scanning, by at least one scanning device of the mobile cleaning device, the space in which the mobile cleaning device is located;
determining, by a computing device of the mobile cleaning device, a series of conceptual physical nodes within the space in which the mobile cleaning device is located;
calculating, by the computing device, a shortest pathway from an origin node of the series of conceptual physical nodes, to each of the other conceptual physical nodes, and back to the origin node;
determining, by the computing device, an activation schedule of at least one array of UV lights on the mobile cleaning device for when the mobile cleaning device is located at each of the conceptual physical nodes; and
traversing the mobile cleaning device along the shortest pathway from the origin node, and activating the at least one array of UV lights at each node of the plurality of conceptual physical nodes according to the activation schedule to degrade pathogens in the space in which the mobile cleaning device is located.

17. The method of claim 16, wherein activating the at least one array of UV lights includes activating at least two arrays of UV lights arranged in a modular configuration about the mobile cleaning device, and wherein the at least two arrays of UV lights are activated to sterilize at least one of a floor, a wall, a ceiling, or an object positioned in the space in which the mobile cleaning device is located.

18. The method of claim 16, wherein the mobile cleaning device includes a proximity sensor configured to detect movement of an object or person within a predetermined distance from the mobile cleaning device, and the method further includes activating a warning light on the mobile cleaning device when the object or person is detected within the predetermined distance from the mobile cleaning device.
